(19) United States
(12) Patent Application Publication
(10) Pub. No.: US 2025/0014693 A1
(43) Pub. Date: Jan. 9, 2025

(54) MEDICAL DATA MANAGEMENT SYSTEM BASED ON PATCH-TYPE WEARABLE DEVICE AND METHOD OF OPERATING THE SAME

(71) Applicant: ODN Co., Ltd., Seoul (KR)

(72) Inventor: Sang Youl RHEE, Seoul (KR)

(73) Assignee: ODN Co., Ltd., Seoul (KR)

(21) Appl. No.: 18/625,796

(22) Filed: Apr. 3, 2024

(30) Foreign Application Priority Data
Jul. 4, 2023 (KR) 10-2023-0086556

(51) Int. Cl. G16H 10/60; G16H 20/10; G16H 40/60

(52) U.S. Cl. CPC G16H 10/60; G16H 20/10; G16H 40/60

(57) ABSTRACT

The present disclosure relates to a medical data management system and a method of operating the same. The medical data management system according to one embodiment includes a data collector for collecting biometric data corresponding to at least one of times before and after surgery or procedure from a patch-type wearable device attached to a user; a data provider for providing medical data corresponding to the collected biometric data to a pre-set terminal of a medical institution; and a data manager for receiving prescription data corresponding to the medical data from the terminal of the medical institution and updating the received prescription data in an electronic medical record (EMR) database of the medical institution.

### CROSS-REFERENCE TO RELATED APPLICATION

This application claims priority to Korean Patent Application No. 10-2023-0086556, filed on Jul. 4, 2023, in the Korean Intellectual Property Office, the disclosure of which is incorporated herein by reference.

### BACKGROUND OF THE DISCLOSURE

### Field of the Disclosure

The present disclosure relates to a medical data management system and a method of operating the same, and more particularly, to a technical idea that monitors the biometric information of a user (patient) based on a patch-type wearable device and stores and manages medical data according to the monitoring results.

### Description of the Related Art

Before performing surgery/procedure that require anesthesia or local anesthesia, such as liposuction and indications, a medical institution checks the patient's exact condition to accurately determine whether the surgery/procedure will proceed. Through this process, the possibility of side effects and complications due to surgery/procedure can be minimized.

However, in a conventional procedure or surgery process, the patient's condition is confirmed only by relying on the patient's questionnaire data and a simple examination process (blood test, etc.) conducted at a hospital. Accordingly, medical staff has limitations in objectively and accurately determining whether surgery/procedure will proceed.

### RELATED ART DOCUMENTS

### Patent Documents

Korean Patent Application Publication No. 10-2014-0050374, “PATCH-TYPE ELECTROCARDIOGRAM MEASURING DEVICE AND METHOD OF OPERATING THE SAME”

### SUMMARY OF THE DISCLOSURE

Therefore, the present disclosure has been made in view of the above problems, and it is an object of the present disclosure to provide a medical data management system capable of more easily collecting the biometric data of a user using a patch-type wearable device and a method of operating the same.

It is another object of the present disclosure to provide a medical data management system that allows medical staff to more accurately determine whether to perform a procedure or surgery on a user by providing medical data corresponding to the biometric data of the user to the medical staff and a method of operating the same.

It is yet another object of the present disclosure to provide a medical data management system capable of improving the accuracy of treatment and prescription for a user by medical staff by providing comparative results of biometric data before and after procedure or surgery of the user and a method of operating the same.

In accordance with one aspect of the present disclosure, provided is a medical data management system including a data collector for collecting biometric data corresponding to at least one of times before and after surgery or procedure from a patch-type wearable device attached to a user; a data provider for providing medical data corresponding to the collected biometric data to a pre-set terminal of a medical institution; and a data manager for receiving prescription data corresponding to the medical data from the terminal of the medical institution and updating the received prescription data in an electronic medical record (EMR) database of the medical institution.

According to one aspect, the patch-type wearable device may be attached to at least one of a surgery or procedure region, thoracoabdominal region, arm, and hand of the user to collect the biometric data and may provide the collected biometric data to the data collector through at least one of a pre-set terminal, App service, and Web service of the user.

According to one aspect, the biometric data may include data corresponding to at least one biometric information among electrocardiogram, heart rate, oxygen saturation, temperature, and respiration.

According to one aspect, the data collector may collect the biometric data every pre-set period for the at least one time.

According to one aspect, the data provider may provide, to a terminal of the medical institution, at least one medical data of first medical data including first biometric data collected corresponding to the time before surgery or procedure and second medical data including second biometric data collected corresponding to the time after surgery or procedure.

According to one aspect, the second medical data may include comparison result data based on difference between the second biometric data and the first biometric data.

According to one aspect, the data manager may generate medical service information corresponding to the received prescription data and provide the generated medical service information to the pre-set terminal of the user.

In accordance with another aspect of the present disclosure, provided is a medical data management method including collecting, in a data collector, biometric data corresponding to at least one of times before and after surgery or procedure from a patch-type wearable device attached to a user; providing, in a data provider, medical data corresponding to the collected biometric data to a pre-set terminal of a medical institution; and receiving, in a data manager, prescription data corresponding to the medical data from the terminal of the medical institution and updating the received prescription data in an electronic medical record (EMR) database of the medical institution.

According to one aspect, the patch-type wearable device may be attached to at least one of a surgery or procedure region, thoracoabdominal region, arm, and hand of the user to collect the biometric data and may provide the collected biometric data to the data collector through at least one of a pre-set terminal, App service, and Web service of the user.

According to one aspect, in the providing, the data provider may provide, to a terminal of the medical institution, at least one medical data of first medical data including first biometric data collected corresponding to the time before surgery or procedure and second medical data including second biometric data collected corresponding to the time after surgery or procedure.

According to one aspect, in the providing, the data provider may provide the second medical data including comparison result data based on difference between the second biometric data and the first biometric data.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a diagram explaining a medical data management system according to one embodiment;

FIG. 2 is a diagram explaining a patch-type wearable device according to one embodiment;

FIG. 3 is a flowchart explaining a medical data management method according to one embodiment; and

FIG. 4 is a flowchart explaining the medical data management method according to one embodiment in more detail.

### DETAILED DESCRIPTION OF THE DISCLOSURE

Specific structural and functional descriptions of embodiments according to the concept of the present disclosure disclosed herein are merely illustrative for the purpose of explaining the embodiments according to the concept of the present disclosure. Furthermore, the embodiments according to the concept of the present disclosure can be implemented in various forms and the present disclosure is not limited to the embodiments described herein.

The embodiments according to the concept of the present disclosure may be implemented in various forms as various modifications may be made. The embodiments will be described in detail herein with reference to the drawings. However, it should be understood that the present disclosure is not limited to the embodiments according to the concept of the present disclosure, but includes changes, equivalents, or alternatives falling within the spirit and scope of the present disclosure.

The terms such as “first” and “second” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the teachings of the present disclosure.

It should be understood that when an element is referred to as being “connected to” or “coupled to” another element, the element may be directly connected or coupled to the other element or intervening elements may be present. In contrast, when an element is referred to as being “directly connected to” or “directly coupled to” another element, there are no intervening elements present. Other words used to describe the relationship between elements or layers should be interpreted in a like fashion (e.g., “between,” versus “directly between,” “adjacent,” versus “directly adjacent,” etc.).

The terms used in the present specification are used to explain a specific exemplary embodiment and not to limit the present inventive concept. Thus, the expression of singularity in the present specification includes the expression of plurality unless clearly specified otherwise in context. Also, terms such as “include” or “comprise” should be construed as denoting that a certain characteristic, number, step, operation, constituent element, component or a combination thereof exists and not as excluding the existence of or a possibility of an addition of one or more other characteristics, numbers, steps, operations, constituent elements, components or combinations thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, preferred embodiments of the present disclosure will be described in detail with reference to the accompanying drawings. However, the scope of the present disclosure is not limited by these embodiments. Like reference numerals in the drawings denote like elements.

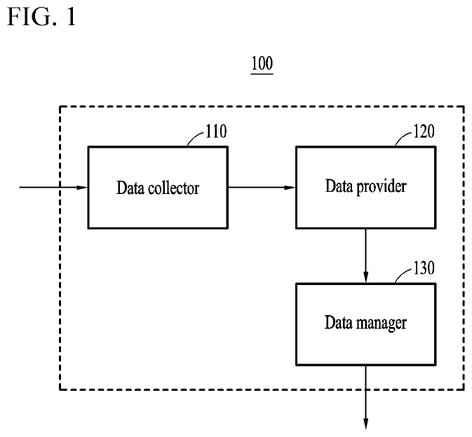

FIG. 1 is a diagram explaining a medical data management system according to one embodiment.

Referring to FIG. 1, a medical data management system 100 according to one embodiment may more easily collect the biometric data of a user using a patch-type wearable device.

In addition, the medical data management system 100 may allow medical staff to more accurately determine whether to perform a procedure or surgery on a user by providing medical data corresponding to the biometric data of the user to the medical staff.

In addition, the medical data management system 100 may improve the accuracy of treatment and prescription for a user by medical staff by providing comparative results of biometric data before and after procedure or surgery of the user.

The medical data management system 100 may include a data collector 110, a data provider 120, and a data manager 130.

The data collector 110 according to one embodiment may collect biometric data corresponding to at least one of times before and after surgery or procedure from a patch-type wearable device attached to a user.

For example, the user may be a patient undergoing surgery or procedure for which anesthesia or local anesthesia is applied.

Specifically, the medical data management system 100 may be applied to patients who need a procedure or surgery due to liposuction and indications, without being limited thereto. In addition to liposuction and indications, the medical data management system 100 may be applied to patients in various cases using medical institutions, such as patients undergoing various surgeries (procedures), non-hospitalized patients, and patients receiving day surgery clinic to minimize hospitalization. According to one aspect, the data collector 110 may collect biometric data from the time a patch-type wearable device is attached to a user prior to surgery or procedure to the time the patch-type wearable device is separated from the user, as first biometric data corresponding to the time before the surgery or procedure is performed.

In addition, the data collector 110 may collect biometric data from the time a patch-type wearable device is attached to a user after surgery or procedure to the time the patch-type wearable device is separated from the user, as second biometric data corresponding to the time after the surgery or procedure is performed.

According to one aspect, the patch-type wearable device may be attached to at least one of a surgery or procedure region, thoracoabdominal region, arm, and hand of a user to collect biometric data. However, the attachment site of the patch-type wearable device according to one embodiment is not limited thereto, and the patch-type wearable device may be attached to any part of the body from which the biometric data of the user may be collected.

The collected biometric data may be provided to the data collector 110 through at least one of a pre-set terminal, App service, and Web service of the user.

For example, the biometric data may include data corresponding to at least one biometric information among electrocardiogram, heart rate, oxygen saturation, temperature, and respiration.

According to one aspect, the data collector 110 may collect biometric data every pre-set period for the at least one time. Preferably, the data collector 110 may collect biometric data in real time through at least one of a terminal, App service, and Web service of a user.

The data provider 120 according to one embodiment may provide medical data corresponding to the collected biometric data to a pre-set terminal of a medical institution.

In addition, the data manager 130 according to one embodiment may receive prescription data corresponding to the medical data from the terminal of the medical institution and update the received prescription data to an electronic medical record (EMR) database of the medical institution.

According to one aspect, the data provider 120 may provide, to a terminal of the medical institution, at least one medical data of first medical data including first biometric data collected corresponding to the time before surgery or procedure and second medical data including second biometric data collected corresponding to the time after surgery or procedure.

For example, the first medical data may be data obtained by preprocessing the first biometric data based on a pre-set medical form, and the second medical data may be data obtained by preprocessing the second biometric data based on a pre-set medical form.

According to one aspect, the data provider 120 may compare at least one of an electrocardiogram value, a body surface temperature value, and a respiration value included in the first biometric data with a pre-set risk judgment threshold value and generate risk information based on the comparative results.

In addition, the data provider 120 may provide the first medical data including the generated risk information to the terminal of the medical institution.

That is, the data provider 120 may allow medical staff to more accurately determine whether to perform a procedure or surgery on a user by providing the first medical data including the risk information to the medical staff.

According to one aspect, the second medical data may include comparison result data based on the difference between the second biometric data and the first biometric data.

For example, the comparison result data may include at least one difference value among the electrocardiogram difference value, body surface temperature difference value, and respiration difference value between the second biometric data and the first biometric data.

According to one aspect, the data provider 120 may generate side effect/complication judgment information based on at least one difference value and a pre-set judgment threshold value and may provide second medical data including the generated side effect/complication judgment information to the terminal of the medical institution.

That is, the data provider 120 may allow medical staff to more easily determine whether side effects or complications have occurred in a user who has undergone a procedure or surgery by providing the second medical data including the side effect/complication judgment information to the medical staff. Based on the judgment results, the data provider 120 may provide prescriptions to the user more quickly and accurately.

According to one aspect, the data manager 130 may generate medical service information corresponding to the received prescription data and provide the generated medical service information to a pre-set terminal of the user.

For example, the data manager 130 may provide the second medical data including at least one of a patient consultation service, an educational material provision service, and a visit notification service to a user through at least one of a terminal, App service, and Web service of the user.

In addition, the medical data management system 100 may be linked to a pre-set digital treatment management system to induce prescription for digital therapeutics and provide medical service information according to the prescription of digital therapeutics to the user.

Specifically, the data provider 120 may generate a list of first digital therapeutics corresponding to at least one biometric data among the first biometric data and the second biometric data and provide at least one medical data among the first medical data and second medical data including the generated first digital therapeutics to the terminal of the medical institution.

In addition, when the data manager 130 receives prescription data including one digital therapeutics among a plurality of digital therapeutics included in a list of first digital therapeutics from the terminal of the medical institution, the data manager 130 may generate medical service information corresponding to the digital therapeutics included in prescription data and provide the generated medical service information to the pre-set terminal of the user.

According to one aspect, the data provider 120 may extract a list of second digital therapeutics from a list of first digital therapeutics based on the comparison result data based on the difference between the second biometric data and the first biometric data, and may provide the extracted second digital therapeutics to the terminal of the medical institution.

Specifically, the data provider 120 may monitor changes in the difference value of electrocardiogram, difference value of body surface temperature, and difference value of respiration based on the comparison result data, and may extract a plurality of digital therapeutics corresponding to monitoring results from a list of first digital therapeutics as a list of second digital therapeutics and provide the extracted digital therapeutics to the terminal of the medical institution. Through this process, the data provider 120 may induce more accurate prescriptions of digital therapeutics by medical staff.

For example, when the difference value of each parameter (e.g., electrocardiogram, body surface temperature, respiration) on the comparison result data of the user is outside the pre-set normal range value during the pre-set time, the data provider 120 may extract a plurality of digital therapeutics corresponding to parameters outside the normal range as a list of second digital therapeutics.

According to one aspect, the data manager 130 may collect evaluation information about each digital therapeutics included in a list of first digital therapeutics from medical personnel belonging to the medical institution, and may generate non-recommended information corresponding to digital therapeutics for which the collected evaluation information is less than the pre-set threshold evaluation information. The data provider 120 may extract the remaining digital therapeutics excluding digital therapeutics corresponding to the non-recommended information from a list of first digital therapeutics or a list of second digital therapeutics as a list of third digital therapeutics, and may provide the extracted third digital therapeutics to the terminal of the medical institution.

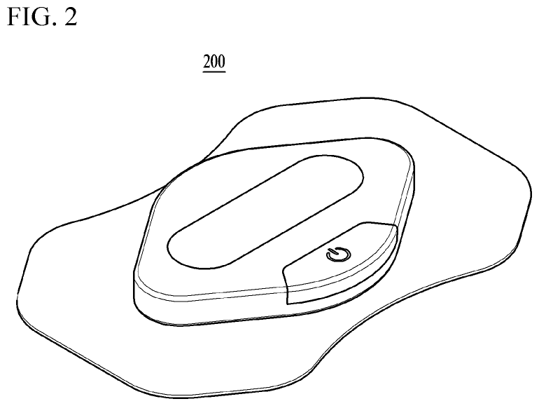

FIG. 2 is a diagram explaining a patch-type wearable device according to one embodiment.

Referring to FIG. 2, a patch-type wearable device 200 according to one embodiment may be attached to the surgery or procedure region, thoracoabdominal region, arm, or hand of a user, may collect data corresponding to at least one biometric information among the electrocardiogram, heart rate, oxygen saturation, temperature, and respiration of the user as the biometric data of the user, and may provide the collected biometric data to the medical data management system according to one embodiment.

For example, the patch-type wearable device 200 may be a disposable device equipped with a small capacity battery, at least one sensor that detects the user's biometric data, and a short-range communication means, and may operate for about 1 to 2 weeks.

According to one aspect, the patch-type wearable device 200 may provide the collected biometric data to the medical data management system according to one embodiment through the user's terminal or equipment equipped with a separate communication means.

Specifically, when the patch-type wearable device 200 is used before the user's surgery or procedure, the patch-type wearable device 200 may assist medical staff in determining whether a user has an underlying disease or infectious disease based on the regularity of the user's biometric data obtained prior to surgery or procedure.

In addition, when the patch-type wearable device 200 is used after the user's surgery or procedure, the patch-type wearable device 200 may monitor the user's biometric data at home (i.e., discharged from hospital) and may quickly respond to the user's risky situations (side effects or complications, etc.).

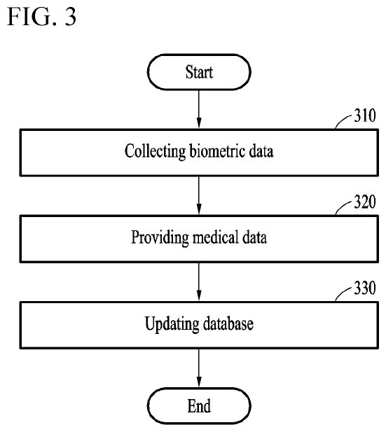

FIG. 3 is a flowchart explaining a medical data management method according to one embodiment.

That is, FIG. 3 is a flowchart explaining a method of operating the medical data management system according to one embodiment described with reference to FIGS. 1 and 2.

Referring to FIG. 3, according to the medical data management method, in step 310, a data collector may collect biometric data corresponding to at least one of times before and after surgery or procedure from a patch-type wearable device attached to a user.

For example, the patch-type wearable device may be attached to at least one of the surgery or procedure region, thoracoabdominal region, arm, and hand of the user to collect biometric data, and may provide the collected biometric data to the data collector through at least one of a pre-set terminal, App service, and Web service of the user.

In addition, the biometric data may include data corresponding to at least one biometric information among electrocardiogram, heart rate, oxygen saturation, temperature, and respiration.

According to one aspect, according to the medical data management method, in step 310, the data collector may collect biometric data every pre-set period for the at least one time.

Next, according to the medical data management method, in step 320, a data provider may provide medical data corresponding to the collected biometric data to a pre-set terminal of a medical institution.

According to one aspect, according to the medical data management method, in step 320, the data provider may provide, to a terminal of the medical institution, at least one medical data of first medical data including first biometric data collected corresponding to the time before surgery or procedure and second medical data including second biometric data collected corresponding to the time after surgery or procedure.

According to one aspect, according to the medical data management method, in step 320, the data provider may provide second medical data including comparison result data based on the difference between second biometric data and first biometric data.

Next, according to the medical data management method, in step 330, the data manager may receive prescription data corresponding to the medical data from the terminal of the medical institution and update the received prescription data to an electronic medical record (EMR) database of the medical institution.

According to one aspect, according to the medical data management method, in step 330, the data manager may generate medical service information corresponding to the received prescription data and provide the generated medical service information to a pre-set terminal of the user.

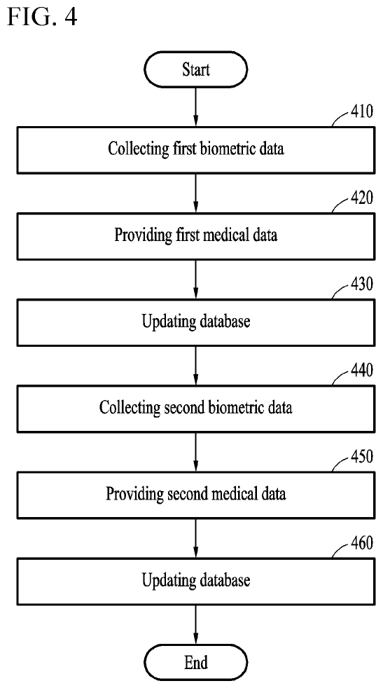

The medical data management method according to one embodiment will be explained in more detail with reference to FIG. 4.

FIG. 4 is a flowchart explaining the medical data management method according to one embodiment in more detail.

Referring to FIG. 4, according to the medical data management method, in step 410, the data collector may collect the first biometric data of a user at the time before procedure or surgery (i.e., time prior to performance of surgery or procedure) from the patch-type wearable device attached to the user.

For example, the first biometric data may include data corresponding to at least one biometric information among the electrocardiogram, heart rate, oxygen saturation, temperature, and respiration of the user detected by the patch-type wearable device at the time before procedure or surgery.

Next, according to the medical data management method, in step 420, the data provider may provide first medical data corresponding to the collected first biometric data to a pre-set terminal of a medical institution.

For example, the first medical data may be medical data of the user including the first biometric data. For example, the first medical data may be data obtained by preprocessing the first biometric data based on a pre-set medical form.

According to one aspect, according to the medical data management method, in step 420, the data provider may compare at least one of an electrocardiogram value, a body surface temperature value, and a respiration value included in the first biometric data with a pre-set risk judgment threshold value and generate risk information based on the comparative results. In addition, the data provider may provide the first medical data including the generated risk information to the terminal of the medical institution.

That is, according to the medical data management method, in step 420, the data provider may allow medical staff to more accurately determine whether to perform a procedure or surgery on the user by providing first medical data including risk information to the medical staff.

According to one aspect, according to the medical data management method, in step 420, the data provider may generate a list of first digital therapeutics corresponding to the first biometric data and provide the first medical data including the generated first digital therapeutics to the terminal of the medical institution.

Next, according to the medical data management method, in step 430, the data manager may receive first prescription data corresponding to the first medical data from the terminal of the medical institution and update the received first prescription data to an electronic medical record (EMR) database of the medical institution.

According to one aspect, according to the medical data management method, in step 430, the data manager may generate medical service information corresponding to the received first prescription data and provide the generated medical service information to the terminal of the user.

For example, the medical service information may include at least one of a patient consultation service, an educational material provision service, and a visit notification service.

According to one aspect, according to the medical data management method, in step 430, when the data manager receives first prescription data including one digital therapeutics among a plurality of digital therapeutics included in a list of first digital therapeutics corresponding to the first biometric data from the terminal of the medical institution, the data manager may generate first medical service information corresponding to the digital therapeutics included in prescription data and provide the generated medical service information to the pre-set terminal of the user.

Next, according to the medical data management method, in step 440, the data collector may collect the second biometric data of the user at the time after surgery or procedure (i.e., time after performance of surgery or procedure) from the patch-type wearable device attached to the user.

For example, the second biometric data may include data corresponding to at least one biometric information among electrocardiogram, heart rate, oxygen saturation, temperature, and respiration of the user detected by the patch-type wearable device at the time after procedure or surgery.

Next, according to the medical data management method, in step 450, the data provider may provide second medical data corresponding to the collected second biometric data to the pre-set terminal of the medical institution.

For example, the second medical data may be medical data of the user including the second biometric data. For example, the second medical data may be data obtained by preprocessing the second biometric data based on a pre-set medical form.

According to one aspect, according to the medical data management method, in step 450, the data provider may provide second medical data including comparison result data based on the difference between the second biometric data and the first biometric data.

For example, the comparison result data may include at least one difference value among the electrocardiogram difference value, body surface temperature difference value, and respiration difference value between the second biometric data and the first biometric data.

According to one aspect, according to the medical data management method, in step 450, the data provider may generate side effect/complication judgment information based on at least one difference value and a pre-set judgment threshold value and may provide second medical data including the generated side effect/complication judgment information to the terminal of the medical institution.

That is, according to the medical data management method, in step 450, the data provider may allow medical staff to more easily determine whether side effects or complications have occurred in a user who has undergone a procedure or surgery by providing the second medical data including the side effect/complication judgment information to the medical staff. Based on the judgment results, the data provider may provide prescriptions to the user more quickly and accurately.

According to one aspect, according to the medical data management method, in step 450, the data provider may generate a list of first digital therapeutics corresponding to the second biometric data and provide the second medical data including the generated first digital therapeutics to the terminal of the medical institution.

Next, according to the medical data management method, in step 460, the data manager may receive second prescription data corresponding to the second medical data from the terminal of the medical institution and update the received second prescription data to an electronic medical record (EMR) database of the medical institution.

According to one aspect, according to the medical data management method, in step 460, the data manager may generate medical service information corresponding to the received second prescription data and provide the generated medical service information to the terminal of the user.

For example, the medical service information may include at least one of a patient consultation service, an educational material provision service, and a visit notification service.

According to one aspect, according to the medical data management method, in step 460, when the data manager receives second prescription data including one digital therapeutics among a plurality of digital therapeutics included in a list of first digital therapeutics corresponding to the second biometric data from the terminal of the medical institution, the data manager may generate second medical service information corresponding to the digital therapeutics included in prescription data and provide the generated medical service information to the pre-set terminal of the user.

In conclusion, when the present disclosure is used, the biometric data of a user may be more easily collected by using a patch-type wearable device.

In addition, when the present disclosure is used, it can be more accurately determined whether to perform a procedure or surgery on a user by providing medical data corresponding to the biometric data of the user to medical staff.

In addition, when the present disclosure is used, the accuracy of treatment and prescription for a user by medical staff may be improved by providing comparative results of biometric data before and after procedure or surgery of the user.

According to one embodiment, the present disclosure can more easily collect the biometric data of a user using a patch-type wearable device.

According to one embodiment, the present disclosure can help medical staff more accurately decide whether to perform a procedure or surgery on a user by providing medical data corresponding to the biometric data of the user to the medical staff.

According to one embodiment, the present disclosure can improve the accuracy of treatment and prescription for a user by medical staff by providing comparative results of biometric data before and after procedure or surgery of the user.

Although the present disclosure has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

[Description of Symbols]

| | |
|---|---|
| 100: MEDICAL DATA | 110: DATA |
| MANAGEMENT SYSTEM | COLLECTOR |
| 120: DATA PROVIDER | 130: DATA MANAGER |

What is claimed is:

1. A medical data management system, comprising:
a data collector for collecting biometric data corresponding to at least one of times before and after surgery or procedure from a patch-type wearable device attached to a user;
a data provider for providing medical data corresponding to the collected biometric data to a pre-set terminal of a medical institution; and
a data manager for receiving prescription data corresponding to the medical data from the terminal of the medical institution and updating the received prescription data in an electronic medical record (EMR) database of the medical institution.

2. The medical data management system according to claim 1, wherein the patch-type wearable device is attached to at least one of a surgery or procedure region, thoracoabdominal region, arm, and hand of the user to collect the biometric data and provides the collected biometric data to the data collector through at least one of a pre-set terminal, App service, and Web service of the user.

3. The medical data management system according to claim 1, wherein the biometric data comprises data corresponding to at least one biometric information among electrocardiogram, heart rate, oxygen saturation, temperature, and respiration.

4. The medical data management system according to claim 1, wherein the data collector collects the biometric data every pre-set period for the at least one time.

5. The medical data management system according to claim 1, wherein the data provider provides, to a terminal of the medical institution, at least one medical data of first medical data comprising first biometric data collected corresponding to the time before surgery or procedure and second medical data comprising second biometric data collected corresponding to the time after surgery or procedure.

6. The medical data management system according to claim 5, wherein the second medical data comprises comparison result data based on difference between the second biometric data and the first biometric data.

7. The medical data management system according to claim 1, wherein the data manager generates medical service information corresponding to the received prescription data and provides the generated medical service information to the pre-set terminal of the user.

8. A medical data management method, comprising:
collecting, in a data collector, biometric data corresponding to at least one of times before and after surgery or procedure from a patch-type wearable device attached to a user;
providing, in a data provider, medical data corresponding to the collected biometric data to a pre-set terminal of a medical institution; and
receiving, in a data manager, prescription data corresponding to the medical data from the terminal of the medical institution and updating the received prescription data in an electronic medical record (EMR) database of the medical institution.

9. The medical data management method according to claim 8, wherein the patch-type wearable device is attached to at least one of a surgery or procedure region, thoracoabdominal region, arm, and hand of the user to collect the biometric data and provides the collected biometric data to the data collector through at least one of a pre-set terminal, App service, and Web service of the user.

10. The medical data management method according to claim 8, wherein, in the providing, the data provider provides, to a terminal of the medical institution, at least one medical data of first medical data comprising first biometric data collected corresponding to the time before surgery or procedure and second medical data comprising second biometric data collected corresponding to the time after surgery or procedure.

11. The medical data management method according to claim 10, wherein, in the providing, the data provider provides the second medical data comprising comparison result data based on difference between the second biometric data and the first biometric data.
